(19) United States
(12) Patent Application Publication
(10) Pub. No.: US 2005/0234076 A1
(43) Pub. Date: Oct. 20, 2005

(54) Triazolopyrimidines

(76) Inventors: Olaf Gebauer, Koln (DE); Nico Jorg Greul, Leichlingen (DE); Ulrich Heinemann, Leichlingen (DE); Fritz Maurer, Lauffen (DE); Bernd-Wieland Kruger, Bergisch-Gladbach (DE); Hans-Ludwig Elbe, Wuppertal (DE); Herbert Gayer, Monheim (DE); Ralf Dunken, Monheim (DE); Arnd Voerste, Koln (DE); Stefan Hillebrand, Neuss (DE); Christiane Boie, Leichlingen (DE); Ulrike Wachendorff-Neumann, Neuwied (DE); Astrid Mauler-Machnik, Leichlingen (DE); Karl-Heinz Kuck, Langenfeld (DE)

Correspondence Address: BAYER CROPSCIENCE LP;Patent Department, 100 BAYER ROAD, PITTSBURGH, PA, 15205-9741, US

(21) Appl. No.: 10/511,821

(22) PCT Filed: Apr. 14, 2003
(86) PCT No.: PCT/EP03/03833

§ 371 Date: May 11, 2005

(30) Foreign Application Priority Data
Apr. 26, 2002 (DE) 10218592.1

(51) Int. Cl. A61K 31/519; C07D 487/04

(52) U.S. Cl. 514/259.31; 544/263

(57) ABSTRACT

Novel triazolopyrimidines of the formula 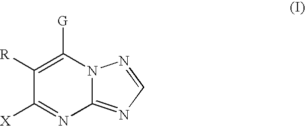 in which G, R and X are as defined in the description, a process for preparing these substances and their use for controlling unwanted microorganisms.

The present invention relates to novel triazolopyrimidines, to a process for their preparation and to their use for controlling unwanted microorganisms.

It is already known that certain triazolopyrimidines have fungicidal properties (cf. EP-A-0 550 113, WO 94/20501, EP-A-0 613 900, U.S. Pat. No. 5,612,345, EP-A-0 834 513, FR-A-2 784 991, WO 98/46607 and WO 98/46608). However, in many cases the activity of these compounds is unsatisfactory.

This invention now provides novel triazolopyrimidines of the formula 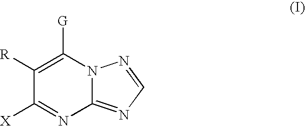
in which
G represents optionally substituted, mono- or polycyclic saturated, unsaturated or aromatic heterocyclyl which is attached via a nitrogen atom, where this nitrogen atom is attached in the heterocycle to a further nitrogen or oxygen atom and where the heterocycle optionally contains one or two further oxygen, nitrogen and/or sulphur atoms, but where no two oxygen atoms are directly adjacent,
R represents aryl which is optionally mono- to pentasubstituted and
X represents halogen,
and acid addition salts of those compounds of the formula (I) in which
G represents optionally substituted, mono- or polycyclic saturated or unsaturated heterocyclyl which is attached via a nitrogen atom, where this nitrogen atom is attached in the heterocycle to a further nitrogen atom and where the heterocycle optionally contains one or two further oxygen, nitrogen and/or sulphur atoms, but where no two oxygen atoms are directly adjacent.

Depending on the substitution pattern, the compounds according to the invention can, if appropriate, be present as mixtures of different possible isomeric forms, in particular of stereoisomers, such as, for example, E- and Z-, threo- and erythro- and also optical isomers, and, if appropriate, also of tautomers. If the substituents on the two atoms in R which are adjacent to the point of attachment are different, the compounds in question may be present in a particular stereoisomeric form, i.e. as atropisomers.

Furthermore, it has been found that the triazolopyrimidines of the formula (I) are obtained when
dihalotriazolopyrimidines of the formula 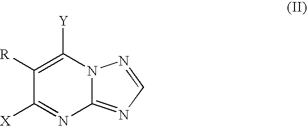
   in which
R and X are as defined above and
Y represents halogen,
are reacted with heterocycles of the formula
   G-H (III)
   in which
G is as defined above,
or with acid addition salts of heterocycles of the formula (III),
if appropriate in the presence of a diluent and if appropriate in the presence of an acid acceptor.

Finally, it has been found that the triazolopyrimidines of the formula (I) and their acid addition salts are highly suitable for controlling unwanted microorganisms. In particular, they have strong fungicidal activity and can be used both in crop protection and in the protection of materials.

Surprisingly, the triazolopyrimidines of the formula (I) according to the invention have considerably better microbicidal activity than the constitutionally more similar prior-art compounds of the same direction of action.

The formula (I) provides a general definition of the triazolopyrimidines according to the invention.
G preferably represents mono- or bicyclic saturated, unsaturated or aromatic heterocyclyl having a total of up to 12 members which is attached via a nitrogen atom, where each nitrogen atom is attached in the heterocycle to a further nitrogen or oxygen atom and where the heterocycle optionally contains one or two further oxygen, nitrogen and/or sulphur atoms, but where no two oxygen atoms are directly adjacent,
   where the heterocycles may be mono- to trisubstituted by identical or different substituents from the group consisting of cyano, halogen, alkyl having 1 to 4 carbon atoms, haloalkyl having 1 to 4 carbon atoms and 1 to 9 identical or different halogen atoms or by alkoxycarbonyl having 1 to 4 carbon atoms in the alkoxy group,
R preferably represents phenyl, which is optionally mono- to tetrasubstituted by identical or different substituents from the group consisting of:
   halogen, cyano, nitro, amino, hydroxyl, formyl, carboxy, carbamoyl, thio-carbamoyl;
   in each case straight-chain or branched alkyl, alkoxy, alkylthio, alkylsulphinyl or alkylsulphonyl having in each case 1 to 6 carbon atoms;
   in each case straight-chain or branched alkenyl or alkenyloxy having in each case 2 to 6 carbon atoms;
   in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;
   in each case straight-chain or branched haloalkenyl or haloalkenyloxy having in each case 2 to 6 carbon atoms and 1 to 11 identical or different halogen atoms;
   in each case straight-chain or branched alkylamino, dialkylamino, alkyl-carbonyl, alkylcarbonyloxy, alkoxycarbonyl, alkylsulphonyloxy, hydrox-iminoalkyl or alkoximinoalkyl having in each case 1 to 6 carbon atoms in the individual alkyl moieties;
   in each case doubly attached alkylene having 3 or 4 carbon atoms or dioxyalkylene having 1 or 2 carbon atoms, or cycloalkyl having 3 to 6 carbon atoms, each of which radicals is optionally mono- or polysubstituted by identical or different substituents from the group consisting of halogen, straight-chain or branched alkyl having 1 to 4 carbon atoms and straight-chain or branched haloalkyl having 1 to 4 carbon atoms and 1 to 9 identical or different halogen atoms,
X preferably represents fluorine, chlorine or bromine.

Particular preference is given to those compounds of the formula (I) in which
G represents a heterocyclyl radical of the formula 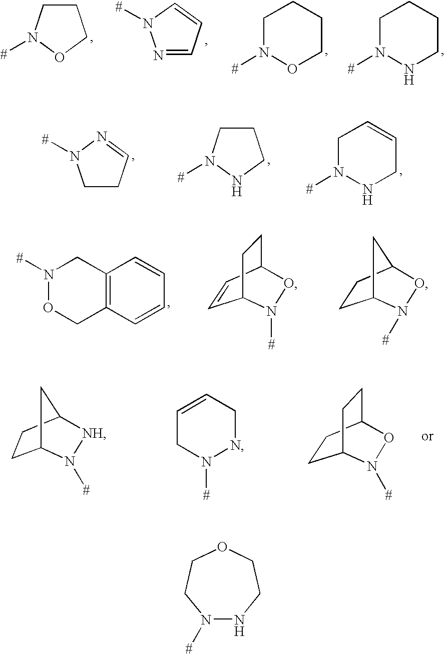
   where # denotes the point of attachment and where each of the radicals may be mono- to trisubstituted by identical or different substituents from the group consisting of cyano, fluorine, chlorine, methyl, ethyl, methoxycarbonyl and ethoxycarbonyl,
R represents phenyl which may be mono- to tetrasubstituted by identical or different substituents from the group consisting of fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulphinyl, ethylsulphinyl, methylsulphonyl or ethyl-sulphonyl, allyloxy, propargyloxy, trifluoromethyl, trifluoroethyl, difluoro-methoxy, trifluoromethoxy, difluorochloromethoxy, trifluoroethoxy, difluoro-methylthio, difluorochloromethylthio, trifluoromethylthio, trifluoromethyl-sulphinyl, trifluoromethylsulphonyl, trichloroethynyloxy, trifluoroethynyloxy, chloroallyloxy, iodopropargyloxy, methylamino, ethylamino, n- or i-propyl-amino, dimethylamino, diethylamino, acetyl, propionyl, acetyloxy, methoxy-carbonyl, ethoxycarbonyl, hydroximinomethyl, hydroximinoethyl, methox-iminoethyl, ethoximinoethyl, cyclopropyl, cyclobutyl, cyclopentyl or cyclohexyl,
   trimethylene (propane-1,3-diyl) which is attached in the 2,3-position or 3,4-position, methylenedioxy or ethylenedioxy, each of which is optionally mono- or polysubstituted by identical or different substituents from the group consisting of fluorine, chlorine, methyl, trifluoromethyl, ethyl, n- or i-propyl, and
X represents bromine or chlorine.

Very particular preference is given to those compounds of the formula (I) in which
G and X have the meanings which have already been mentioned as being particularly preferred and
R represents phenyl which may be mono- to tetrasubstituted by identical or different substituents from the group consisting of fluorine, chlorine, trifluoromethyl, trifluoromethoxy and trifluoromethylthio, or
R represents the radical of the formula

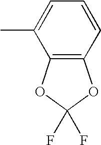

Preference is also give to acid addition salts of those compounds of the formula (I) in which G represents mono- or bicyclic saturated or unsaturated heterocyclyl having up to 12 ring members which is attached via a nitrogen atom, where this nitrogen atom is attached in the heterocycle to a further nitrogen atom and where the heterocycle optionally contains one or two further oxygen, nitrogen and/or sulphur atoms, but where no two oxygen atoms are directly adjacent, where the heterocycles may be mono- to trisubstituted by identical or different substituents from the group consisting of cyano, halogen, alkyl having 1 to 4 carbon atoms, haloalkyl having 1 to 4 carbon atoms and 1 to 9 identical or different halogen atoms or by alkoxycarbonyl having 1 to 4 carbon atoms in the alkoxy group, and R and X have those meanings which have been mentioned as being preferred for these radicals.

Acids which may be added preferably include hydrohalic acids, such as, for example, hydrochloric acid and hydrobromic acid, in particular hydrochloric acid, furthermore phosphoric acid, nitric acid, mono- and bifunctional carboxylic acids and hydroxycarboxylic acids, such as, for example, acetic acid, maleic acid, succinic acid, fumaric acid, tartaric acid, citric acid, salicyclic acid, sorbic acid and lactic acid, and also sulphonic acids, such as, for example, p-toluene sulphonic acid, 1,5-naphtha-lenedisulphonic acid, saccharin and thiosaccharin.

Particular preference is given to salts which are formed by addition of hydrochloric acid, phosphoric acid, p-toluenesulphonic acid, 1,5-naphthalenedisulphonic acid or saccharin to triazolopyrimidines of the formula (I) in which
G represents a heterocyclyl radical of the formula 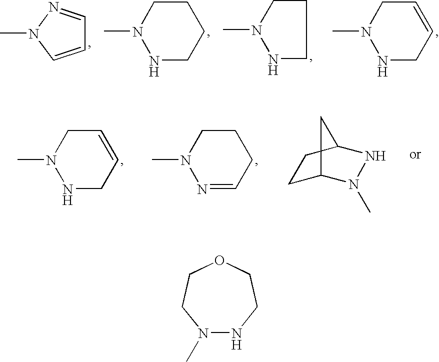
   where each of these radicals may be mono- to trisubstituted by identical or different substituents from the group consisting of cyano, fluorine, chlorine, methyl, ethyl, methoxycarbonyl and ethoxycarbonyl, and
R and X have those meanings which have been mentioned as being particularly preferred for these radicals.

The radical definitions mentioned above can be combined with one another as desired. Moreover, individual meanings may not apply.

The general or preferred radical definitions given above apply both to the end products of the formula (I) and, correspondingly, to the starting materials or intermediates required in each case for the preparation.

Using 5,7-dichloro-6-(2,4,6-trifluorophenyl)[1,2,4]triazolo[1,5a]pyrimidine and 3-methylisoxazolidine hydrochloride as starting materials, the course of the process according to the invention can be illustrated by the following scheme below.

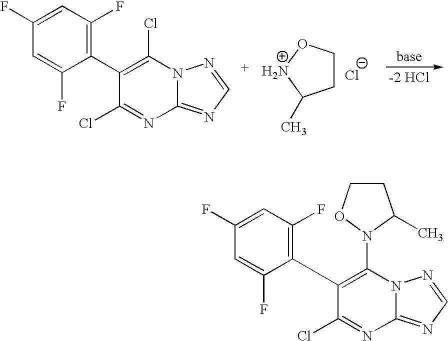

The formula (II) provides a general definition of the dihalotriazolopyrimidines required as starting materials for carrying out the process according to the invention. In this formula (II), R and X preferably or in particular have those meanings which have already been mentioned in connection with the description of the compounds of the formula (I) according to the invention as being preferred and particularly preferred, respectively, for R and X. Y preferably represents fluorine, chlorine or bromine, in particular fluorine or chlorine.

The dihalotriazolopyrimidines of the formula (II) are known or can be prepared by known methods (cf., for example, U.S. Pat. No. 5,612,345).

The formula (III) provides a general definition of the heterocycles furthermore required as starting materials for carrying out the process according to the invention. In this formula (III), G preferably or in particular has that meaning which has already been given in connection with the description of the compounds of the formula (I) according to the invention as being preferred or particularly preferred, respectively, for G.

The heterocycles of the formula (III) are known or can be prepared by known methods (cf., for example, J. Chem. Soc. 1942, 432; Can. J. Chem. (1976), 54(6), 867-70; Tetrahedron Lett. (1993), 34(36), 5673-6; Tetrahedron Lett. (1973), 30, 2859-2862).

When carrying out the process according to the invention, the heterocycles of the formula (III) can also be used in the form of their acid addition salts. Acid addition salts which are preferred here are those compounds which are formed by addition of the acids which have already been mentioned in connection with the description of the acid addition salts according to the invention, to heterocycles of the formula (III). Preference is given to hydrochlorides and acetates of heterocycles of the formula (III).

Suitable diluents for carrying out the process according to the invention are all inert organic solvents. Preference is given to using aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl-t-butyl ether, methyl-t-amyl ether, dioxane, tetrahydrofuran, 1,2-dimethoxyethane, 1,2-diethoxyethane or anisole; amides, such as N,N-dimethylformamide, N,N-dimethylacetamide, N-methylformanilide, N-methyl-pyrrolidone or hexamethylphosphoric triamide; esters, such as methyl acetate or ethyl acetate; sulphoxides, such as dimethylsulphoxide; sulphones, such as sulpholane.

Suitable acid acceptors for carrying out the process according to the invention are all customary acid binders. Preference is given to using ammonia or tertiary amines, such as trimethylamine, triethylamine, tributylamine, N,N-dimethylaniline, N,N-dimethylbenzylamine, pyridine, N-methylpiperidine, N-methylmorpholine, N,N-di-methylaminopyridine, diazabicyclooctane (DABCO), diazabicyclononene (DBN) or diazabicycloundecene (DBU). Also suitable are alkaline earth metal or alkali metal hydrides, hydroxides, amides, alkoxides, acetates, carbonates or bicarbonates, such as, for example, sodium hydride, sodium amide, sodium methoxide, sodium ethoxide, potassium tert-butoxide, sodium hydroxide, potassium hydroxide, sodium acetate, potassium acetate, calcium acetate, sodium carbonate, potassium carbonate, potassium bicarbonate and sodium bicarbonate.

When carrying out the process according to the invention, the reaction temperatures can be varied within a relatively wide range. In general, the process is carried out at temperatures between 0° C. and 150° C., preferably between 0° C. and 80° C.

The process according to the invention is generally carried out under atmospheric pressure. However, it is also possible to operate under elevated pressure of up to 10 bar or under reduced pressure of up to 0.1 bar.

When carrying out the process according to the invention, in general from 0.5 to 10 mol, preferably from 0.8 to 2 mol, of a compound of the formula (II) are employed per mole of dihalotriazolopyrimidine of the formula (II). Work-up is carried out by customary methods.

For preparing acid addition salts of triazolopyrimidines of the formula (I), preference is given to using those acids which have already been mentioned in connection with the description of the acid addition salts according to the invention as preferred acids.

The acid addition salts of the compounds of the formula (I) can be obtained in a simple manner by customary methods for forming salts, for example by dissolving a compound of the formula (I) in a suitable inert solvent and adding the acid, for example hydrochloric acid, and they can be isolated in a known manner, for example by filtration, and, if appropriate, be purified by washing with an inert organic solvent.

The substances according to the invention have potent microbicidal activity and can be employed for controlling undesirable microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:
Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
Pythium species, such a s, for example, Pythium ultimum;
Phytophthora species, such as, for example, Phytophthora infestans;
Pseudoperonospora species, such as, for example, Pseudoperonospora humuli or Pseudoperonospora cubensis;
Plasmopara species, such as, for example, Plasmopara viticola;
Bremia species, such as, for example, Bremia lactucae;
Peronospora species, such as, for example, Peronospora pisi or P. brassicae;
Erysiphe species, such as, for example, Erysiphe graminis;
Sphaerotheca species, such as, for example, Sphaerotheca fuliginea;
Podosphaera species, such as, for example, Podosphaera leucotricha;
Venturia species, such as, for example, Venturia inaequalis;
Pyrenophora species, such as, for example, Pyrenophora teres or P. graminea (conidia form: Drechslera, syn: Helminthosporium);
Cochliobolus species, such as, for example, Cochliobolus sativus (conidia form: Drechslera, syn: Helminthosporium);
Uromyces species, such as, for example, Uromyces appendiculatus;
Puccinia species, such as, for example, Puccinia recondita;
Sclerotinia species, such as, for example, Sclerotinia sclerotiorum;
Tilletia species, such as, for example, Tilletia caries;
Ustilago species, such as, for example, Ustilago nuda or Ustilago avenae;
Pellicularia species, such as, for example, Pellicularia sasakii;
Pyricularia species, such as, for example, Pyricularia oryzae;
Fusarium species, such as, for example, Fusarium culmorum;
Botrytis species, such as, for example, Botrytis cinerea;
Septoria species, such as, for example, Septoria nodorum;
Leptosphaeria species, such as, for example, Leptosphaeria nodorum;
Cercospora species, such as, for example, Cercospora canescens;
Alternaria species, such as, for example, Alternaria brassicae; and
Pseudocercosporella species, such as, for example, Pseudocercosporella herpotrichoides.

The active compounds according to the invention also have very good fortifying action in plants. Accordingly, they can be used for mobilizing the plant's defences against attack by undesirable microorganisms.

In the present context, plant-fortifying (resistance-inducing) substances are to be understood as meaning those substances which are capable of stimulating the defence system of plants such that, when the treated plants are subsequently inoculated with undesirable microorganisms, they show substantial resistance against these mircroorganisms.

In the present case, undesirable microorganisms are to be understood as meaning phytopathogenic fungi, bacteria and viruses. Accordingly, the substances according to the invention can be used to protect plants for a certain period after the treatment against attack by the pathogens mentioned. The period for which protection is provided generally extends over 1 to 10 days, preferably 1 to 7 days, after the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be used with particularly good results for controlling diseases in viticulture and in the cultivation of fruit and vegetables, such as, for example, against Botrytis, Venturia and Alternaria species, or rice diseases, such as, for example against Pyricularia species.

The active compounds according to the invention are also suitable for increasing the yield of crops. In addition, they show reduced toxicity and are well tolerated by plants.

At certain concentrations and application rates, the active compounds according to the invention can also be used as herbicides, for influencing plant growth and for controlling animal pests. If appropriate, they can also be used as intermediates and precursors for the synthesis of further active compounds.

According to the invention, it is possible to treat all plants and parts of plants. Plants are to be understood here as meaning all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multi-layer coating.

In the protection of materials, the compounds according to the invention can be employed for protecting industrial materials against infection with, and destruction by, undesired microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes), and against slime organisms and algae.

Microorganisms of the following genera may be mentioned as examples:
Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is, liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be used as such or in their formulations, also in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Examples of suitable mixing components are the following:

Fungicides:
aldimorph, ampropylfos, ampropylfos-potassium, andoprim, anilazine, azaconazole, azoxystrobin,
benalaxyl, benodanil, benomyl, benzamacril, benzamacril-isobutyl, bialaphos, binapacryl, biphenyl, bitertanol, blasticidin-S, bromuconazole, bupirimate, buthiobate,
calcium polysulphide, carpropamide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,
debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,
edifenphos, epoxiconazole, etaconazole, ethirimol, etridiazole,
famoxadon, fenapanil, fenarimol, fenbuconazole, fenfuram, fenhexamide, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulphamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, fluoxastrobin,
guazatine,
hexachlorobenzene, hexaconazole, hymexazole,
imazalil, imibenconazole, iminoctadine, iminoctadine albesilate, iminoctadine triacetate, iodocarb, ipconazole, iprobenfos (IBP), iprodione, iprovalicarb, irumamycin, isoprothiolane, isovaledione,
kasugamycin, kresoxim-methyl, copper preparations, such as: copper hydroxide, copper naphthenate, copper oxychloride, copper sulphate, copper oxide, oxine-copper and Bordeaux mixture,
mancopper, mancozeb, maneb, meferimzone, mepanipyrim, mepronil, metalaxyl, metconazole, methasulphocarb, methfuroxam, metiram, metomeclam, metsulphovax, mildiomycin, myclobutanil, myclozolin,
nickel dimethyldithiocarbamate, nitrothal-isopropyl, nuarimol,
ofurace, oxadixyl, oxamocarb, oxolinic acid, oxycarboxim, oxyfenthiin,
paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur, prothioconazole,
quinconazole, quintozene (PCNB), quinoxyfen,
sulphur and sulphur preparations,
tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole,
uniconazole,
validamycin A, vinclozolin, viniconazole,
zarilamide, zineb, ziram and also
Dagger G,
OK-8705,
OK-8801,
α-(1,1 -dimethylethyl)-β-(2-phenoxyethyl)-1H-1,2,4-triazole-1-ethanol,
α-(2,4-dichlorophenyl)-β-fluoro-β-propyl-1H-1,2,4-triazole-1-ethanol,
α-(2,4-dichlorophenyl)-α-methoxy-α-methyl-1H-1,2,4-triazole-1-ethanol,
α-(5-methyl-1,3-dioxan-5-yl)-β-[[4-(trifluoromethyl)phenyl]methylene]-1H-1,2,4-triazole-1-ethanol,
(5RS,6RS)-6-hydroxy-2,2,7,7-tetramethyl-5-(1H-1,2,4-triazol-1-yl)-3-octanone,
(E)-α-(methoxyimino)-N-methyl-2-phenoxyphenylacetamide,
1-(2,4-dichlorophenyl)-2-(1H-1,2,4-triazol-1-yl)ethanone O-(phenylmethyl)oxime,
1-(2-methyl-1-naphthalenyl)-1H-pyrrole-2,5-dione,
1-(3,5-dichlorophenyl)-3-(2-propenyl)-2,5-pyrrolidinedione,
1-[(diiodomethyl)sulphonyl]-4-methylbenzene,
1-[[2-(2,4-dichlorophenyl)-1,3-dioxolan-2-yl]methyl]-1H-imidazole,
1-[[2-(4-chlorophenyl)-3-phenyloxiranyl]methyl]-1H-1,2,4-triazole,
1-[1-[2-[(2,4-dichlorophenyl)methoxy]phenyl]ethenyl]-1H-imidazole,
1-methyl-5-nonyl-2-(phenylmethyl)-3-pyrrolidinole,
2′,6′-dibromo-2-methyl-4′-trifluoromethoxy-4′-trifluoromethyl-1,3-thiazole-5-carboxanilide,
2,6-dichloro-5-(methylthio)-4-pyrimidinylthiocyanate,
2,6-dichloro-N-(4-trifluoromethylbenzyl)benzamide,
2,6-dichloro-N-[[4-(trifluoromethyl)phenyl]methyl]benzamide,
2-(2,3,3-triiodo-2-propenyl)-2H-tetrazole,
2-[(1-methylethyl)sulphonyl]-5-(trichloromethyl)-1,3,4-thiadiazole,
2-[[6-deoxy-4-O-(4-O-methyl-β-D-glycopyranosyl)-α-D-glucopyranosyl]amino]-4-methoxy-1H-pyrrolo[2,3-d]pyrimidine-5-carbonitrile,
2-aminobutane,
2-bromo-2-(bromomethyl)pentanedinitrile,
2-chloro-N-(2,3-dihydro-1,1,3-trimethyl-1H-inden-4-yl)-3-pyridinecarboxamide,
2-chloro-N-(2,6-dimethylphenyl)-N-(isothiocyanatomethyl)acetamide,
2-phenylphenol (OPP),
3,4-dichloro-1-[4-(difluoromethoxy)phenyl]-1H-pyrrole-2,5-dione,
3,5-dichloro-N-[cyano[(1-methyl-2-propynyl)oxy]methyl]benzamide,
3-(1,1-dimethylpropyl-1-oxo-1H-indene-2-carbonitrile,
3-[2-(4-chlorophenyl)-5-ethoxy-3-isoxazolidinyl]pyridine,
4-chloro-2-cyano-N,N-dimethyl-5-(4-methylphenyl)-1H-imidazole-1-sulphonamide,
4-methyltetrazolo[1,5-a]quinazolin-5(4H)-one,
8-hydroxyquinoline sulphate,
9H-xanthene-2-[(phenylamino)carbonyl]-9-carboxylic hydrazide,
bis-(1-methylethyl)-3-methyl-4-[(3-methylbenzoyl)oxy]-2,5-thiophenedicarboxylate,
cis-1-(4-chlorophenyl)-2-(1H-1,2,4-triazol-1-yl)cycloheptanol,
cis-4-[3-[4-(1,1-dimethylpropyl)phenyl-2-methylpropyl]-2,6-dimethylmorpholine hydrochloride,
ethyl [(4-chlorophenyl)azo]cyanoacetate,
potassium hydrogen carbonate,
methanetetrathiol sodium salt,
methyl 1-(2,3-dihydro-2,2-dimethyl-1H-inden-1-yl)-1H-imidazole-5-carboxylate,
methyl N-(2,6-dimethylphenyl)-N-(5-isoxazolylcarbonyl)-DL-alaninate,
methyl N-(chloroacetyl)-N-(2,6-dimethylphenyl)-DL-alaninate,
N-(2,6-dimethylphenyl)-2-methoxy-N-(tetrahydro-2-oxo-3-furanyl)acetamide,
N-(2,6-dimethylphenyl)-2-methoxy-N-(tetrahydro-2-oxo-3-thienyl)acetamide,
N-(2-chloro-4-nitrophenyl)-4-methyl-3-nitrobenzenesulphonamide,
N-(4-cyclohexylphenyl)-1,4,5,6-tetrahydro-2-pyrimidineamine,
N-(4-hexylphenyl)-1,4,5,6-tetrahydro-2-pyrimidineamine,
N-(5-chloro-2-methylphenyl)-2-methoxy-N-(2-oxo-3-oxazolidinyl)acetamide,
N-(6-methoxy-3-pyridinyl)cyclopropanecarboxamide,
N-[2,2,2-trichloro-1-[(chloroacetyl)aminolethyl]benzamide,
N-[3-chloro-4,5-bis-(2-propinyloxy)phenyl]-N′-methoxymethaneimidamide,
N-formyl-N-hydroxy-DL-alanine-sodium salt,
O,O-diethyl [2-(dipropylamino)-2-oxoethyl]ethylphosphoramidothioate,
O-methyl S-phenyl phenylpropylphosphoramidothioate,
S-methyl 1,2,3-benzothiadiazole-7-carbothioate,
spiro[2H]-1-benzopyrane-2,1′(3′H)-isobenzofuran]-3′-one,
4-[3,4-dimethoxyphenyl)-3-(4-fluorophenyl)acryloyl]morpholine.

Bactericides:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:
abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin,
Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben,
cadusafos, carbaryl, carbofuran, carbophenothion, carbosulphan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine,
deltamethrin, demeton M, demeton S, demeton-S-methyl, diafenthiuron, diazinon, dichlorvos, dicofol, diflubenzuron, dimethoat, dimethylvinphos, diofenolan, disulphoton, docusat-sodium, dofenapyn,
eflusilanate, emamectin, empenthrin, endosulphan, Entomopfthora spp., esfenvalerate, ethiofencarb, ethion, ethoprophos, etofenprox, etoxazole, etrimfos,
fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb,
granulosis viruses,
halofenozide, HCH, heptenophos, hexaflumuron, hexythiazox, hydroprene,
imidacloprid, indoxacarb, isazofos, isofenphos, isoxathion, ivermectin,
nuclear polyhedrosis viruses,
lambda-cyhalothrin, lufenuron,
malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos,
naled, nitenpyram, nithiazine, novaluron,
omethoate, oxamyl, oxydemethon M,
Paecilomyces fumosoroseus, parathion A, parathion M, permethrin, phenthoate, phorat, phosalone, phosmet, phospharnidon, phoxim, pirimicarb, pirimiphos A, pirimiphos M, profenofos, promecarb, propargite, propoxur, prothiofos, prothoat, pymetrozine, pyraclofos, pyresmethrin, pyrethrum, pyridaben, pyridathion, pyrimidifen, pyriproxyfen,
quinalphos,
ribavirin,
salithion, sebufos, silafluofen, spinosad, spirodiclofen, sulphotep, sulprofos,
tau-fluvalinate, tebufenozide, tebufenpyrad, tebupirimiphos, teflubenzuron, tefluthrin, temephos, temivinphos, terbufos, tetrachlorvinphos, tetradifon theta-cypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,
vamidothion, vaniliprole, Verticillium lecanii,
YI 5302
zeta-cypermethrin, zolaprofos
(1R-cis)-[5-(phenylmethyl)-3-furanyl]methyl-3-[(dihydro-2-oxo-3(2H)furanylidene)-methyl]-2,2-dimethylcyclopropanecarboxylate,
(3-phenoxyphenyl)methyl-2,2,3,3-tetramethylcyclopropanecarboxylate,
1-[(2-chloro-5-thiazolyl)methyl]tetrahydro-3,5-dimethyl-N-nitro-1,3,5-triazine-2(1H)-imine,
2-(2-chloro-6-fluorophenyl)-4-[4-(1,1-dimethylethyl)phenyl]-4,5-dihydro-oxazole,
2-(acetyloxy)-3-dodecyl-1,4-naphthalenedione,
2-chloro-N-[[[4-(1-phenylethoxy)phenyl]amino]carbonyl]benzamide,
2-chloro-N-[[[4-(2,2-dichloro-1,1-difluoroethoxy)phenyl]amino]-carbonyl]benzamide,
3-methylphenyl propylcarbamate,
4-[4-(4-ethoxyphenyl)-4-methylpentyl]-1-fluoro-2-phenoxybenzene,
4-chloro-2-(1,1-dimethylethyl)-5-[[2-(2,6-dimethyl-4-phenoxyphenoxy)ethyl]thio]-3(2H)-pyridazinone,
4-chloro-2-(2-chloro-2-methylpropyl)-5-[(6-iodo-3-pyridinyl)methoxy]-3(2H)-pyridazinone,
4-chloro-5-[(6-chloro-3-pyridinyl)methoxy]-2-(3,4-dichlorophenyl)-3(2H)-pyridazinone,
Bacillus thuringiensis strain EG-2348,
[2-benzoyl-1-(1,1-dimethylethyl)hydrazinobenzoic acid,
2,2-dimethyl-3-(2,4-dichlorophenyl)-2-oxo-1-oxaspiro[4.5]dec-3-en-4-yl butanoate,
[3-[(6-chloro-3-pyridinyl)methyl)-2-thiazolidinylidene]cyanamide,
dihydro-2-(nitromethylene)-2H-1,3-thiazine-3(4H)-carboxaldehyde,
ethyl [2-[[1,6-dihydro-6-oxo-1-(phenylmethyl)-4-pyridazinyl]oxy]ethyl]carbamate,
N-(3,4,4-trifluoro-1-oxo-3-butenyl)glycine,
N-(4-chlorophenyl)-3-[4-(difluoromethoxy)phenyl]-4,5-dihydro-4-phenyl-1H-pyrazole-1-carboxamide,
N-[(2-chloro-5-thiazolyl)methyl]-N′-methyl-N″-nitroguanidine,
N-methyl-N′-(1-methyl-2-propenyl)-1,2-hydrazinedicarbothioamide,
N-methyl-N′-2-propenyl-1,2-hydrazinedicarbothioamide,
O,O-diethyl [2-(dipropylamino)-2-oxoethyl]ethylphosphoramidothioate,
N-cyanomethyl-4-trifluoromethylnicotinamide,
3,5-dichloro-1-(3,3-dichloro-2-propenyloxy)-4-[3-(5-trifluoromethylpyridin-2-yloxy)propoxy]benzene.

A mixture with other known active compounds, such as herbicides, or with fertilizers and growth regulators, is also possible.

In addition, the compounds of the formula (I) according to the invention also have very good antimycotic activity. They have a very broad antimycotic activity spectrum in particular against dermatophytes and yeasts, moulds and diphasic fungi (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The list of these fungi by no means limits the mycotic spectrum covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low-volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application | rates are generally between 0.1 and 10,000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10,000 g/ha, preferably between 1 and 5000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are understood as meaning plants with novel properties (“traits”) which are grown by conventional cultivation, by mutagenesis or by recombinant DNA techniques. These may be cultivars, breeds biotypes or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which are actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits which are also particularly emphasized are the increased resistance of plants to fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which cultivars will be developed and/or marketed in the future.

The plants listed can be treated according to the invention in a particularly advantageous manner with the compounds of the formula I or the active compound mixtures according to the invention. The preferred ranges stated above for the active compounds or mixtures also apply to the treatment of these plants. Particular emphasis is given to the treatment of plants with the compounds specifically mentioned in the present text.

Preparation and use of the compounds according to the invention are illustrated by the examples below.

### PREPARATION EXAMPLES

### EXAMPLE 1

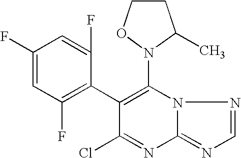

Under argon, 180 mg (0.56 mMol) of 5,7-dichloro-6-(2,4,6-trifluorophenyl)-[1,2,4]triazolo[1,5-a]pyrimidine, 120 mg (0.97 mMol) of 3-methylisoxazolidine hydrochloride and 335 mg of potassium carbonate are stirred in 10 ml of acetonitrile at room temperature for 18 hours. 10 ml of water are added to the reaction mixture and the organic phase is separated off, washed with 10 ml of saturated ammonium chloride solution, dried over sodium sulphate and concentrated under reduced pressure. The residue is chromatographed on silica gel using petroleum ether/ethyl acetate (10:1). This gives 250 mg (49% of theory) of 5-chloro-6-(2,4,6-trifluorophenyl)-7-(3-methyl-2-isoxazolidinyl)[1,2,4]triazolo[1,5-a]pyrimidine.

HPLC: logP=2.76

The compounds of the formula

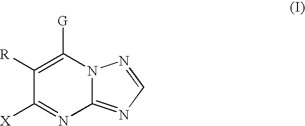

listed in Table 1 below are also obtained analogously to Example 1 and in accordance with the statements in the general description of the process.

TABLE 1

| Ex. | | | | | m.p.: |
|---|---|---|---|---|---|
| No. | X | G | R | logP | (° C.) |
| 2 | Cl | N-isoxazolidinyl | 2,6-difluorophenyl | 2.07 | |
| 3 | Cl | 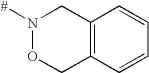 | 2,6-difluorophenyl | 3.33 | |
| 4 | Cl | 4-cyano-1-pyrazolyl | 2,6-dichloro-4- | 3.68 | |
| | | trifluoromethoxyphenyl | | | |
| 5 | Cl | 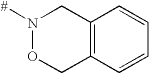 | 2,4,6-trifluorophenyl | 3.53 | |
| 6 | Cl | 1,2-oxazinan-N-yl | 2,4,6-trifluorophenyl | 2.92 | |
| 7 | Cl | N-isoxazolidinyl | 2,4,6-trifluorophenyl | 2.4 | |
| 8 | Cl | 1,2-oxazinan-N-yl | 2-chloro-4-fluorophenyl | 3.11 | |
| 9 | Cl | 1,2-oxazinan-N-yl | 2,4-difluorophenyl | 2.84 | |
| 10 | Cl | tetrahydropyridazin-1-yl | 2,4,6-trifluorophenyl | 2.68 | |
| 11 | Cl | 4,5-dihydropyrazol-1-yl | 2,4,6-trifluorophenyl | 2.34 | 123-26 |
| 12 | Cl | tetrahydropyridazin-1-yl | 2-chloro-6-fluorophenyl | 2.67 | 183-5 |
| 13 | Cl | tetrahydropyridazin-1-yl | 2,2-difluoro-1,3- | 2.97 | 160-6 |
| | | | benzodioxol-4-yl | | |
| 14 | Cl | tetrahydropyridazin-1-yl | 2-chloro-5- | 3.72 | 178-80 |
| | | | trifluoromethylthio- | | |
| | | | phenyl | | |
| 15 | Cl | tetrahydropyridazin-1-yl | 2-chloro-5- | 3.25 | 196-8 |
| | | | trifluoromethylphenyl | | |
| 16 | Cl | tetrahydropyridazin-1-yl | 2-chloro-3- | 3.16 | 142-4 |
| | | | trifluoromethylphenyl | | |
| 17 | Cl | 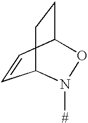 | 2,4,6-trifluorophenyl | 2.82 | |
| 18 | Cl | 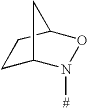 | 2,4,6-trifluorophenyl | 2.75 | |
| 19 | Cl | tetrahydropyridazin-1-yl | 2,6-dichloro-4-tri- | 3.81 | |
| | | | fluoromethoxyphenyl | | |
| 20 | Cl | tetrahydropyridazin-1-yl | 2,6-dichloro-3-fluoro-5- | 3.64 | 208-9 |
| | | | trifluoromethylphenyl | | |
| 21 | Cl | tetrahydropyridazin-1-yl | 2,6-dichlorophenyl | 2.88 | 185-7 |
| 22 | Cl | 4-fluoro-4-methylpyrazolidin-1-yl | 2,4,6-trifluorophenyl | 2.53 | 141-3 |
| 23 | Cl | 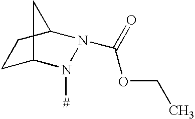 | 2,4,6-trifluorophenyl | 2.8 | 199-02 |
| 24 | Br | tetrahydropyridazin-1-yl | 2,4,6-trifluorophenyl | 2.73 | |
| 25 | Cl | 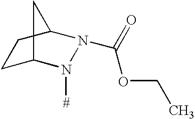 | 2-chloro-6-fluorophenyl | 2.73 | 192-94 |
| 26 | Cl | 3,6-dihydro-2H-pyridazin-1-yl | 2,4,6-trifluorophenyl | 2.53 | 201-03 |
| 27 | Cl | 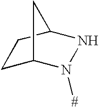 | 2,4,6-trifluorophenyl | 2.65 | 178-80 |
| 28 | Cl | 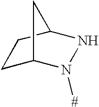 | 2-chloro-6-fluorophenyl | 2.59 | 175-7 |
| 29 | Cl | 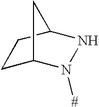 | 2,6-dichlorophenyl | 2.78 | |
| 30 | Cl | 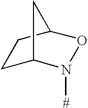 | 2,6-difluorophenyl | 2.53 | |
| 31 | Cl | 1,2-oxazinan-N-yl | 2,6-difluorophenyl | 2.68 | |
| 32 | Cl | 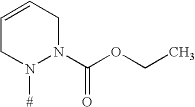 | 2,4,6-trifluorophenyl | 2.82 | Oil |
| 33 | Cl | 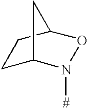 | 2-chlorophenyl | 2.69 | |
| 34 | Cl | 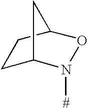 | 2-chloro-6-fluorophenyl | 2.74 | |
| 35 | Cl | 3-methyl-1,2-oxazinan-N-yl | 2,4,6-trifluorophenyl | 3.23 | |
| 36 | Cl | 1,2-oxazinan-N-yl | 2-chloro-6- | 2.92 | |
| | | | fluorophenyl | | |
| 37 | Cl | 1,2-oxazinan-N-yl | 2-chlorophenyl | 2.87 | |
| 38 | Cl | 1,2-oxazinan-N-yl | 2,4-dichlorophenyl | 3.51 | |
| 39 | Cl | 3-methyl-1,2-oxazinan-N-yl | 2,6-difluorophenyl | 2.96 | |
| 40 | Cl | 3-methyl-1,2-oxazinan-N-yl | 2-chloro-4- | 3.59 | |
| | | | fluorophenyl | | |
| 41 | Cl | 3-methyl-1,2-oxazinan-N-yl | 2-chlorophenyl | 3.17 | |
| 42 | Cl | 3-methyl-isoxazolidin-N-yl | 2-chloro-6- | 2.74 | |
| | | | fluorophenyl | | |
| 43 | Cl | 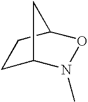 | 2,4-difluorophenyl | 2.67 | |
| 44 | Cl | 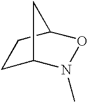 | 2-chloro-4- fluorophenyl | 2.89 | |
| 45 | Cl | 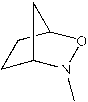 | 2,4-dichlorophenyl | 3.3 | |
| 46 | Cl | 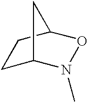 | 2,4-difluoro-6- trifluoromethyl- phenyl | 3.1 | |
| 47 | Cl | 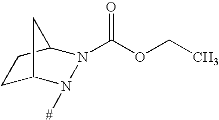 | 2,6-difluorophenyl | 2.57 | 188-90 |
| 48 | Cl | 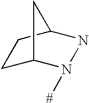 | 2-chloro-4- fluorophenyl | 2.74 | 92-4 decomp. |
| 49 | Cl | 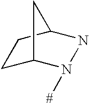 | 2,4,6-trifluorophenyl | 2.88 | 177-80 |
| 50 | Cl | 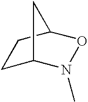 | 2,4,-trifluorophenyl | 2.97 | |
| 51 | Cl | 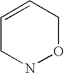 | 2,4,6-tnfluorophenyl | | |
| 52 | Cl | 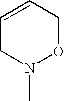 | 2-chlorophenyl | 2.74 | |
| 53 | Cl | 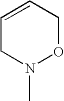 | 2-chloro-4- fluorophenyl | 2.93 | |
| 54 | Cl | 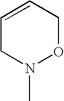 | 2-chloro-6- fluorophenyl | 2.78 | |
| 55 | Cl | 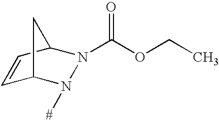 | 2,4,6-trifluorophenyl | 2.79 | paste |
| 56 | Cl | 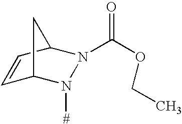 | 2-chloro-6- fluorophenyl | 3.01 | |
| 57 | Cl | 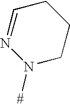 | 2-chloro-6- fluorophenyl | 2.58 | |
| 58 | Cl | 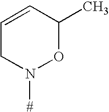 | 2,4,6-trifluorophenyl | 3.12 | |
| 59 | Cl | 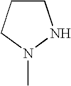 | 2-chloro-6- fluorophenyl | 2.29 | 182-4 |
| 60 | Cl | 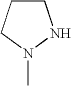 | 2-chloro-4- fluorophenyl | 2.39 | 164-7 |
| 61 | Cl | 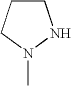 | 2,4,6-trifluorophenyl | 2.32 | |
| 62 | Cl | 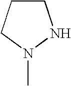 | 2-chlorophenyl | 2.2 | |
| 63 | Cl | tetrahydropyridazin-1-yl | 2,6-difluorophenyl | 2.47 | 188-9 |
| 64 | Cl | tetrahydropyridazin-1-yl | 2,4-difluorophenyl | 2.57 | 180-2 |
| 65 | Cl | tetrahydropyridazin-1-yl | 2-chlorophenyl | 2.61 | 217-8 |
| 66 | Cl | tetrahydropyridazin-1-yl | 2-chloro-4- | 2.8 | 210-2 |
| | | | fluorophenyl | | |
| 67 | Cl | tetrahydropyridazin-1-yl | 2,4-difluoro-6- | 3.06 | 160-1 |
| | | | trifluoromethylphenyl | | |
| 68 | Cl | tetrahydropyridazin-1-yl | 2,6-difluoro-4- | 3.26 | paste |
| | | | trifluoromethylphenyl | | |
| 69 | Cl | tetrahydropyridazin-1-yl | 2,4-dichlorophenyl | 3.22 | 165-6 |
| 70 | Cl | tetrahydropyridazin-1-yl | 4-fluoro-2- | 2.98 | 213-5 |
| | | | trifluoromethylphenyl | | |
| 71 | Cl | 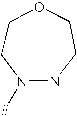 | 2,4,6-trifluorophenyl | 2.18 | |

# in the table above denotes the point of attachment.
*The logP values were determined in accordance with EEC Directive 79/831 Annex V. A8 by HPLC (Gradient method, acetonitrile/0.1% aqueous phosphoric acid)

### Preparation of an Intermediate of the Formula (II)

### EXAMPLE 72

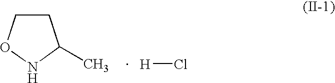

1.83 g (17.4 mmol) of ethyl hydroxycarbonate and 1 g of (4.6 mmol) of 1,3-dibromobutane are added to a solution of 1.6 g of potassium t-butoxide in 40 ml of t-butanol, and the mixture is stirred at 65° C. for 7 hours. The reaction mixture is concentrated under reduced pressure, ether and water are added to the residue and the organic phase is separated off. The aqueous phase is extracted two more times with ether and the combined organic phases are dried over sodium sulphate and concentrated under reduced pressure. This gives 1 g of crude N-ethoxycarbonyl-3-methylisoxazolidine of a purity of about 80% and with a logP value of 1.22.

950 mg of this material are heated under reflux in 10 ml of 16% strength hydrochloric acid for 3 hours. The mixture is concentrated under reduced pressure and triturated three times with 5 ml of methanol and in each case filtered. The combined filtrates are concentrated under reduced pressure. This gives 560 mg of 3-methylethylisoxazolidine hydrochloride having a logP value of 1.22.

### USE EXAMPLES

### EXAMPLE A

Venturia Test (apple)/protective

Solvents: 24.5 parts by weight of acetone
24.5 parts by weight of dimethylacetamide

Emulsifier: 1.0 parts by weight alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and they then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

Active compounds, application rates and test results are shown in the table below.

TABLE A
Venturia Test (apple)/protective

| | Application rate of | |
|---|---|---|
| Active compound | active compound | |
| According to the invention: | in g/ha | Efficacy in % |
| 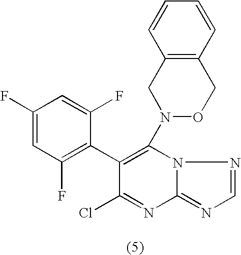 | 100 | 100 |
| 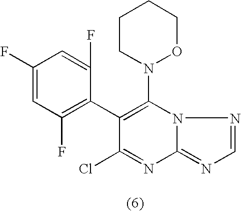 | 100 | 100 |
| 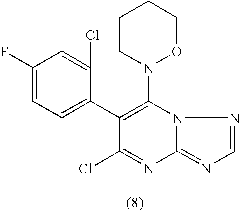 | 100 | 100 |
| 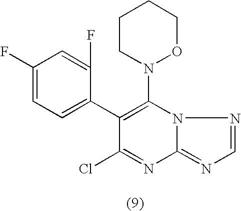 | 100 | 96 |
| 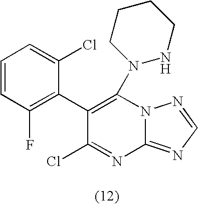 | 100 | 100 |
| 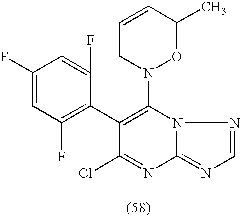 | 100 | 100 |
| 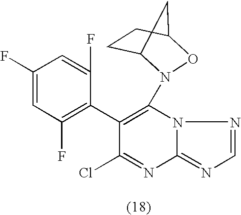 | 100 | 100 |
| 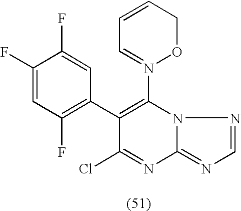 | 100 | 100 |
| 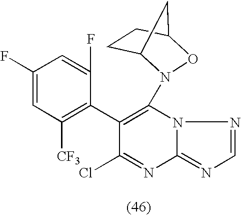 | 100 | 100 |
| 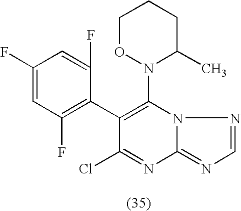 | 100 | 100 |
| 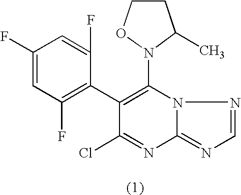 | 100 | 100 |
| 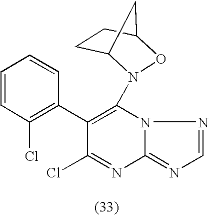 | 100 | 99 |
| 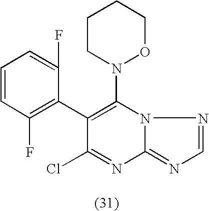 | 100 | 100 |
| 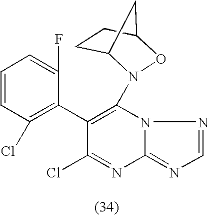 | 100 | 100 |
| 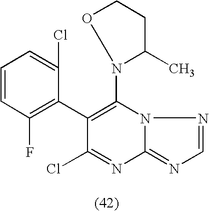 | 100 | 100 |
| 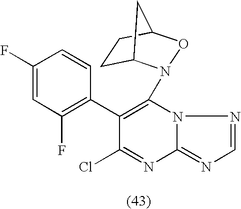 | 100 | 100 |
| 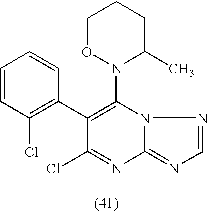 | 100 | 99 |
| 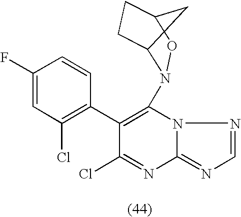 | 100 | 100 |
| 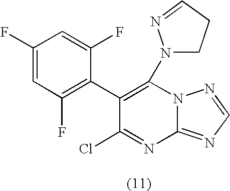 | 100 | 96 |
| 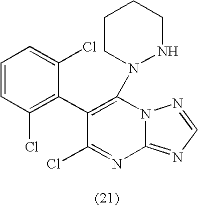 | 100 | 99 |
| 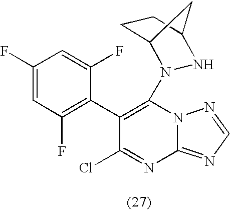 | 100 | 93 |
| 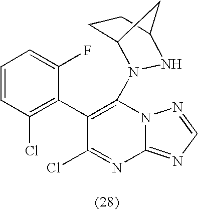 | 100 | 100 |
| 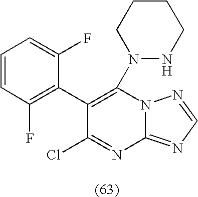 | 100 | 96 |
| 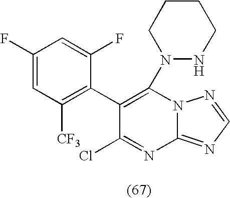 | 100 | 100 |
| 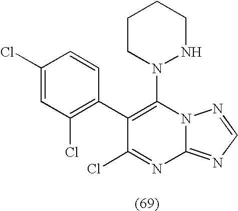 | 100 | 95 |
| 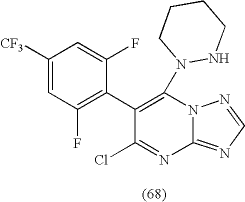 | 100 | 100 |
| 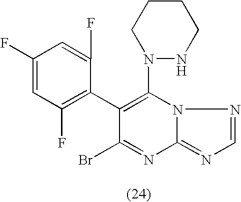 | 100 | 93 |

### EXAMPLE B

Botrytis Test (bean)/protective

Solvents: 24.5 parts by weight of acetone
24.5 parts by of dimethylacetamide

Emulsifier: 1.0 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity.

2 days after the inoculation the size of the infected areas on the leaves is evaluated. 0% means an efficacy which corresponds to that of the control, whereas an efficacy of 100% means that no infection is observed.

Active compounds, application rates and test results are shown in the table below.

TABLE B
Botrytis Test (bean)/protective

| | Application rate of | |
|---|---|---|
| Active compound | active compound in | |
| | g/ha | Efficacy in % |
| 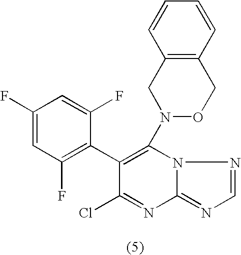 | 500 | 100 |
| 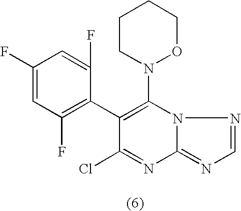 | 500 | 100 |
| 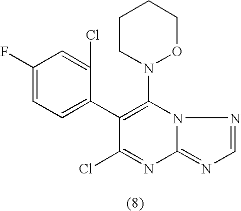 | 500 | 100 |
| 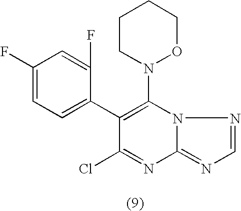 | 500 | 100 |
| 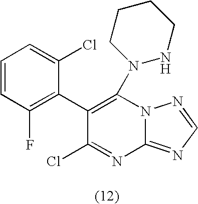 | 500 | 100 |
| 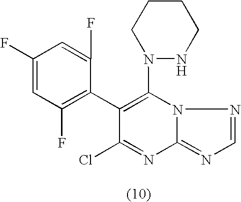 | 500 | 100 |
| 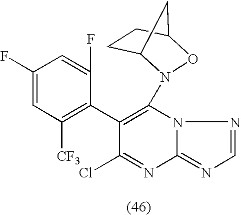 | 500 | 100 |
| 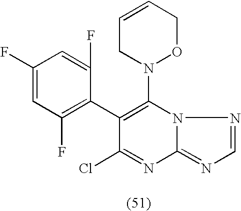 | 500 | 100 |
| 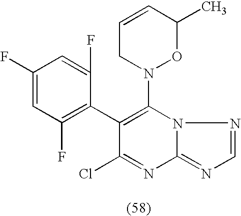 | 500 | 93 |
| 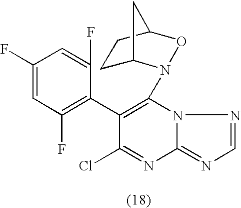 | 500 | 98 |
| 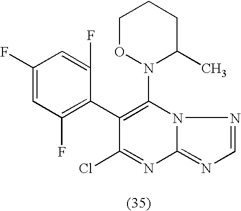 | 500 | 100 |
| 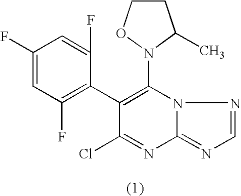 | 500 | 100 |
| 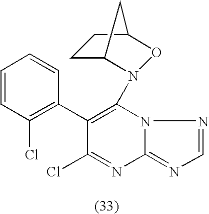 | 500 | 93 |
| 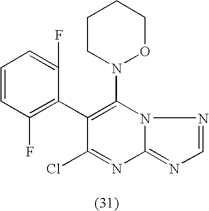 | 500 | 100 |
| 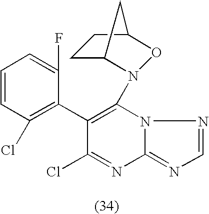 | 500 | 100 |
| 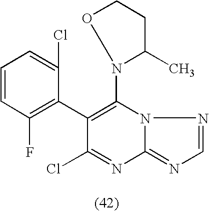 | 500 | 100 |
| 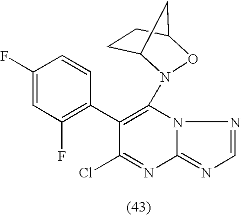 | 500 | 99 |
| 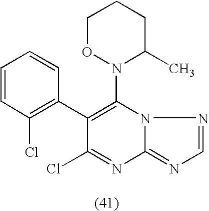 | 500 | 99 |
| 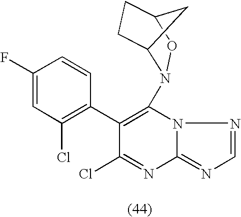 | 500 | 100 |
| 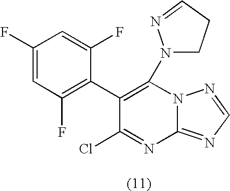 | 500 | 100 |
| 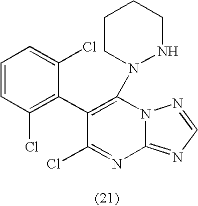 | 500 | 99 |
| 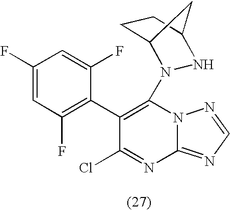 | 500 | 96 |
| 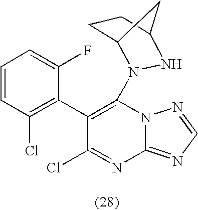 | 500 | 100 |
| 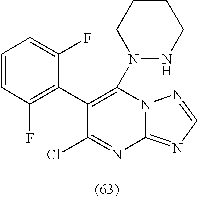 | 500 | 95 |
| 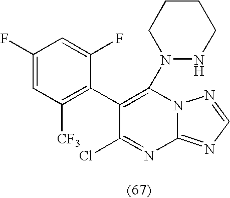 | 500 | 100 |
| 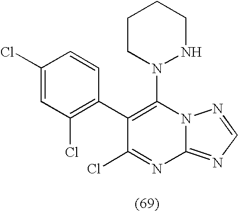 | 500 | 95 |
| 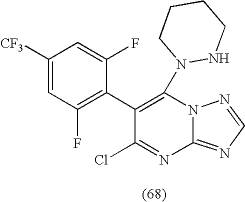 | 500 | 95 |
| 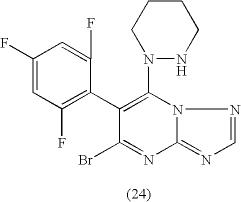 | 500 | 100 |

### EXAMPLE C

Alternaria Test (tomato)/protective

Solvent: 49 parts by weight of N,N-dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the preparation of active compound at the stated application rate. 1 day after the treatment, the plants are inoculated with a spore suspension of Alternaria solani and then remain at 100% relative humidity and 20° C. for 24 h. The plants then remain at 96% relative atmospheric humidity and at a temperature of 20° C.

Evaluation is carried out 7 days after the inoculation. 0% means an efficacy which corresponds to that of the control, whereas an efficacy of 100% means that no infection is observed.

Active compounds, application rates and test results are shown in the table below.

TABLE C
Alternaria Test (tomato)/protective

| | Application rate of | |
|---|---|---|
| Active compound | active compound in | |
| According to the invention: | g/ha Efficacy in % | |
| 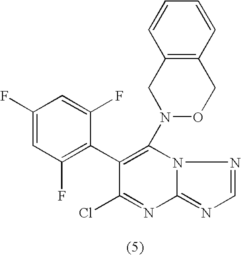 | 750 | 100 |
| 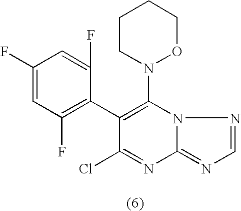 | 750 | 100 |
| 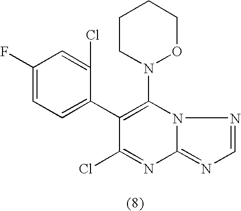 | 750 | 100 |
| 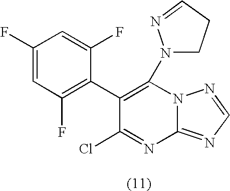 | 750 | 95 |

### EXAMPLE D

Pyricularia Test (rice)/protective

Solvent: 50 parts by weight of N,N-dimethylformamide

Emulsifier: 1 part by weight of alkylaryl polyglycol ether

To produce a suitable preparation of active compound, one part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with an aqueous spore suspension of Pyricularia oryzae. The plants are then placed in a greenhouse at 100% relative atmospheric humidity and 25° C.

Evaluation is carried out 7 days after the inoculation. 0% means an efficacy which corresponds to that of the control, whereas an efficacy of 100% means that no infection is observed.

Active compounds, application rates and test results are shown in the table below.

TABLE D
Pyricularia Test (rice)/protective

| | Application rate of | |
|---|---|---|
| Active compound | active compound | |
| According to the invention: | in g/ha | Efficacy in % |
| 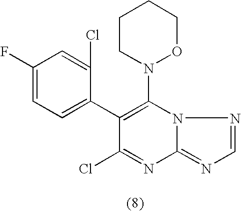 | 500 | 100 |
| 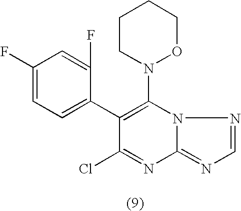 | 500 | 100 |
| 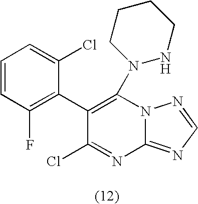 | 500 | 100 |

1. Triazolopyrimidines of the formula 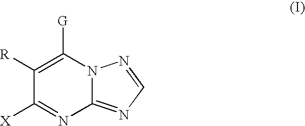
in which
G represents optionally substituted, mono- or polycyclic saturated, unsaturated or aromatic heterocyclyl which is attached via a nitrogen atom, where this nitrogen atom is attached in the heterocycle to a further nitrogen or oxygen atom and where the heterocycle optionally contains one or two further oxygen, nitrogen and/or sulphur atoms, but where no two oxygen atoms are directly adjacent,
R represents aryl which is optionally mono- to pentasubstituted and
X represents halogen,
and acid addition salts of those compounds of the formula (I) in which
G represents optionally substituted, mono- or polycyclic saturated unsaturated or heterocyclyl which is attached via a nitrogen atom, where this nitrogen atom is attached in the heterocycle to a further nitrogen atom and where the heterocycle optionally contains one or two further oxygen, nitrogen and/or sulphur atoms, but where no two oxygen atoms are directly adjacent.

2. Process for preparing triazolopyrimidines of the formula (I) as claimed in claim 1, characterized in that
dihalotriazolopyrimidines of the formula 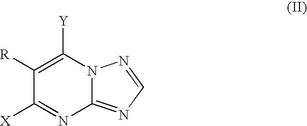
in which
R and X are as defined above and
Y represents halogen
are reacted with heterocycles of the formula
G-H (III)
in which
G is as defined above,
or with acid addition salts of heterocycles of the formula (III)
if appropriate in the presence of a diluent and if appropriate in the presence of an acid acceptor.

3. Microbicidal compositions, characterized in that they comprise at least one triazolopyrimidine of the formula (I) according to claim 1 or an acid addition salt thereof, in addition to extenders and/or surfactants.

4. The use of triazolopyrimidines of the formula (I) according to claim 1 or of their acid addition salts for controlling unwanted microorganisms.

5. Method for controlling unwanted microorganisms, characterized in that triazolopyrimidines of the formula (I) according to claim 1 or their acid addition salts are applied to the unwanted microorganisms and/or their habitat.

6. Process for preparing microbicidal compositions, characterized in that triazolopyrimidines of the formula (I) according to claim 1 or their acid addition salts are mixed with extenders and/or surfactants.
